(19) United States
(12) Patent Application Publication
(10) Pub. No.: US 2003/0049819 A1
(43) Pub. Date: Mar. 13, 2003

(54) Method for the isolation of hydrophobic proteins

(76) Inventors: Folke Tjerneld, Malmo (SE); Ulf Sivars, Stanford, CA (US)

Correspondence Address: Amersham Biosciences Corporation, 800 Centennial Avenue, Piscataway, NJ, 08855, US

(21) Appl. No.: 10/168,710

(22) PCT Filed: Dec. 20, 2000
(86) PCT No.: PCT/EP00/13025

(30) Foreign Application Priority Data
Dec. 27, 1999 (SE) 9904803-5

(51) Int. Cl. C12N 9/00; C07K 1/14

(52) U.S. Cl. 435/183; 530/412

(57) ABSTRACT

A method for separating one or more hydrophobic proteins, for instance membrane proteins such as integral membrane proteins, from a mixture of proteins. The method is characterized in that said mixture is partitioned in a phase system comprising a micelle-enriched aqueous phase (micelle-phase) and a polymer-enriched aqueous phase (polymer phase). At least part of the polymer of the polymer phase carries an affinity ligand that is capable of binding to an affinity structure on at least one of said one or more hydrophobic proteins.

### TECHNICAL FIELD

The present invention concerns a method for the purification of one or more hydrophobic proteins from a mixture of proteins by partitioning the mixture in a phase system comprising an aqueous micelle-enriched phase (micelle phase) and an aqueous polymer-enriched phase (polymer phase). The proteins concerned are primarily membrane proteins, such as integral membrane proteins.

The term “micelle-enriched phase” or “micelle phase” means that the phase contains more micelle-forming agent than polymer. The term “polymer-enriched phase” or “polymer phase” means that the phase contains more polymer than micelle-forming agent. In both cases the comparison is on a weight basis. The phase system may comprise two or more distinct aqueous phases. For simplicity reasons the invention will be described by reference to systems having two phases.

### BACKGROUND ART

Purification of integral membrane proteins is a difficult task. Firstly they are abundant in low levels in complex mixtures of other integral membrane proteins. For E. Coli, only the integral membrane proteins constitute about 6000 proteins (40% of the genome). Secondly they are difficult to overexpress. Thirdly it is difficult to isolate large amount of pure integral membrane proteins in native and stable form with retained structural integrity (1-3). The solubilization of membrane-bound proteins has been reviewed theoretically (4) and isolation of membrane proteins has been subject for several reviews (1,5).

Aqueous two-phase systems have been used in membrane research for separation and subfractionation of membranes, for membrane domain analysis and purification of membrane proteins (7-9). Non-ionic detergents with lower critical solution temperature (cloud point), such as Triton X-114, also form aqueous two-phase systems at temperatures above this temperature (10-12). A two-phase system is formed where a detergent-enriched phase is in equilibrium with a detergent depleted phase. The potential of this system to extract membrane proteins from cytosolic proteins was shown by Bordier (10). The cloud-point extraction technique in detergent systems has since then been developed and applied, especially, as a fast initial purification-step for isolation of membrane proteins from water-soluble proteins and insoluble particles prior to a subsequent high resolution purification method (11,12). As a general rule the membrane proteins have mainly partitioned to the detergent-enriched phase.

The cloud-point extraction technique has some drawbacks. First, the selectivity between different membrane proteins is low. Furthermore, only a few mild non-ionic detergents can be utilized and a specific temperature need to be exceeded for the formation of the two-phase system, both of which can lead to a decreased stability of some membrane proteins. By using a polymer/detergent aqueous two-phase system it is possible both to increase the amount of available detergents and to use lower temperatures (0° C.) (13-15). Phase separation in detergent/polymer/water mixtures has been studied for a number of systems (16-23). In all reported cases, mixtures of non-ionic detergent, non-ionic hydrophilic polymer and water segregate into two phases, one enriched in detergent and the other phase enriched in polymer, and with micelles in each phase. A large range of commonly used non-ionic detergents can form two-phase systems and be used in membrane protein isolation. Typical examples are the Triton series (polyoxyethylene alkyl phenol), alkyl poly(ethyleneoxide) (CₘEOₙ), Tween series (polyoxyethylene sorbitol esters) and alkylglucosides. Phase diagrams of these systems have recently ben presented (15). Recently, we have characterized mechanisms of phase behaviour and protein partitioning in different detergent/polymer aqueous two-phase systems (15). Polymers can be added either to the solubilized (by detergent) membrane proteins or be included into the solubilization buffer together with the detergent. An initial purification of membrane protein can thereby be achieved from the detergent-rich phase a few minutes after the solubilization of the membrane protein is finished. This is advantageous because a fast, partial purification lowers the contact time with proteases and detergent and the extreme partitioning of membrane proteins to the micelle phase makes it possible to reduce the volume without suffering in low recovery. Thus, these systems are of interest for a mild and primary extraction of integral membrane proteins. Se also International patent application WO 9811127 (Ageland H, Nyström L and Tjerneld F) (40).

Some trials have been made to increase selectivity in detergent cloud-point extraction systems most of them dealing with the selective separation of water-soluble proteins (24-26). A few recent studies deal with the selectivity problem for membrane proteins, but only small effects have so far been obtained (14,15,27).

WO 0040598 (Tjerneld F, Persson J, and Johansson H O) (39) describes in the experimental part partitioning bovine serum albumin and apolipoprotein Al between

(a) a first aqueous phase in which a micelle-forming hydrophobically modified ethylene oxide propylene oxide random copolymer (HM-EOPO polymer) is enriched, and

(b) a second aqueous phase in which a non-micelle-forming polymer is enriched.

The application text makes a general statement that the protein to be partitioned may be partitioned to a desired phase by the use of an affinity ligand bound to a polymer.

Metal chelating affinity partitioning has been performed in aqueous two-phase systems for water-soluble proteins (7,29,30) and has been studied in great detail by Arnold and coworkers (31). Immobilized metal affinity chromatography (IMAC) has become a standard technique for purifying recombiant fusion protein (28). See also U.S. Pat. No. 4,7409,304 (Tjerneld F and Johansson G) (41) and U.S. Pat. No. 5,907,035 (Guinn, M R) (42), which suggest affinity ligands in a general form and in form of metal chelates, respectively, for use in two-phase separations.

### OBJECTIVES OF THE INVENTION

A first objective is to provide a method for increasing the selectivity for separating a hydrophobic protein from a mixture of other hydrophobic proteins in phase systems comprising two or more aqueous phases. In particular this objective concerns phase systems, which comprise a micelle-enriched aqueous phase and a polymer-enriched aqueous phase.

Further objectives are to improve the yield and recovery, the purification factor, and the concentration factor in purification methods, which comprise the separation methods given in the preceding paragraph. Primarily the improvement relates to the step utilizing the phase system described.

Still further objectives are to provide methods for the purification of the above-mentioned proteins, which are fast and mild, have high resolution and are suitable for large-scale application.

### THE INVENTION

We have now recognized that these objectives can be accomplished by introducing an affinity ligand on the polymer that is to be partitioned to the polymer phase.

The invention thus is a method for separating one or more hydrophobic proteins from a mixture containing also other proteins that may or may not be hydrophobic. As discussed above the hydrophobic proteins are primarily membrane proteins such as integral membrane proteins. The method is characterized in that it comprises the steps:

(a) providing a sample containing the mixture defined above, said one or more hydrophobic proteins having an affinity structure that is capable of binding to an affinity ligand;

(b) providing a phase system comprising a first aqueous phase in which a micelle-forming agent is enriched (micelle phase) and a second aqueous phase which is in which a polymer is enriched (polymer phase), at least part of said polymer being a conjugate carrying the affinity ligand; and

(c) partitioning the mixture in the phase system so that a complex between said one or more hydrophobic proteins and the conjugate is partitioned to the polymer phase.

A typical subsequent series of steps comprises:

(d) collecting the polymer phase and isolating said one or more hydrophobic proteins carrying the affinity structure; and

(e) if so desired, collecting after step (d) the polymer remaining in the polymer phase including the polymer that carries the affinity ligand, and recycling it into step (b).

An alternative is to collect the polymer phase, release said one or more hydrophobic proteins and incorporate it/them into liposomes.

Between different steps there may be additional steps. The polymer phase, for instance, may preferably be washed before further treatment after step (c). See the experimental part (example 8).

Affinity Structure and Affinity Ligand

The affinity structure may be native to the protein concerned, or may have been inserted by man, for instance by recombinant techniques in form of a peptide affinity tag, or by synthetic means.

In principle any pair of affinity ligand and corresponding affinity structure may be utilized in the invention. Well-known pairs of affinity structures/ligands that have been used in the context of partitioning methods and/or affinity chromatography are: (a) antibodies and antigens/haptens; (b) lectins and carbohydrate structures; (c) IgG binding proteins and IgG; (d) chelates and chelating structures; (e) complementary nucleic acids; (f) biotin and strepavidin etc. Affinity members also include entities participating in catalytic reactions, for instance enzymes, enzyme substrates, cofactors, cosubstrates etc, and chemically produced mimetics of biomolecules. Positively and negatively groups suitable for anion or cation exchange are also of interest as affinity ligands (primary, secondary, tertiary and quaternary ammonium, sulphonate, sulphate, phosphonate, phosphate, carboxy etc groups).

The polymer that carries an affinity ligand is called a conjugate.

Providing a Mixture of Integral Membrane Proteins (Step (a))

The sample contains the mixture of proteins and may be obtained by lysis and solubilization of cells and cell parts. The cells may be a natural occurring type of cell or a recombinant cell line comprising, for instance, a gene encoding the particular integral membrane protein/proteins one is looking for. The solubilized proteins may be used directly in the method according to the invention or may alternatively be pretreated to obtain a fraction, which is enriched with respect to hydrophobic proteins that are to be separated in the process of the invention. A preferred variant for enrichment is to partition the crude solubilized composition in a phase system comprising a micelle-enriched aqueous phase and a micelle-poor aqueous phase which may or may not contain a water-miscible polymer that is incompatible with the micelle-forming agent of the micelle-enriched phase. The micelle-forming agent is typically a detergent but may also be a micelle-forming polymer. In this system the hydrophobic proteins, for instance integral membrane proteins, are mainly partitioned to the micelle-enriched phase while the water-soluble proteins are going to the micelle-poor phase. The micelle-enriched phase may then be used as sample and subsequently mixed with a suitable polymer and conjugate and, if necessary, also with other ingredients to provide the phase system which is provided in step (b) and utilized in step (c).

Providing the Phase System (Step (b))

Components of this phase system may partially derive from the sample provided in step (a). See above. Typically the micelle-forming agent constitutes 1-30%, such as 5-20%, of the micelle phase and the polymer 1-30%, such as 5-20% (w/w), of the polymer phase. Percentages are in w/w.

In order to create distinct phases the micelle-forming agent and the polymer used is selected to be incompatible with each other.

Typical micelle-forming agents are water-soluble (hydrophilic) and non-ionic and are capable of forming two phases when present as an aqueous solution in the presence of the polymer used.

One kind of typical micelle-forming agents are non-ionic detergents, i.e. amphiphilic molecules that have one or more hydrophobic parts and one or more hydrophilic parts and are capable of forming micelle structures in water solutions. Examples are polyoxyethylene alkyl phenol (the Triton series), alkyl poly(ethylene oxide) (CₘEₙ), polyoxyethylene sorbitol esters (Tween series), alkylglucosides etc. See above.

Another kind of micelle-forming agents are so called micelle-forming water-soluble polymers (hydrophilic). Examples of this kind of micelle-forming agents can, for instance, be found among ethylene oxide propylene oxide copolymers have been hydrophobically modified. See also WO 0040598 (Tjerneld F, Persson J, and Johansson H O).

The concentration of the micelle-forming agent typically is above the critical micelle concentration (CMC) in the micelle phase but mostly so also in the polymer phase.

The micelle-forming agent may or may not be thermoseparating.

Typical polymers that define the polymer phase are water-miscible (i.e. hydrophilic) and primarily non-ionic although they may also carry electrically charged groups and/or affinity ligands. The polymer may be thermoseparating. One group of suitable polymers has polysaccharide structure, for instance dextran, starch derivatives and cellulose derivatives. Typical derivatives are alkylated and/or hydroxyalkylated forms of starch and cellulose. Other examples contain identical or different monomer units selected amongst ethylene oxide, propylene oxide, N-substituted acryl amides and N-substituted methacryl amide etc.

At least a part of the polymer defining the polymer phase is in form of a conjugate comprising an affinity ligand as discussed above. The synthesis of conjugates is done according to well known techniques for forming covalent conjugates between a polymer and an affinity ligand. The degree of substitution (DS) for affinity ligands may vary within wide limits. It should be determined on a case to case bases and will depend on at least one variable such as the hydrophobic protein to be separated, kind of polymer, micelle-forming agent used, molecular weight of the polymer etc.

The expression “providing a phase system comprising . . . ” means that the conjugate is incorporated into the phase system in one or more of the following ways:

(i) as part of the sample,

(ii) as part of the phase system provided or

(iii) after the sample has been incorporated into the phase system.

In the phase system there may be present additional conjugates that differ with respect to kind of affinity ligands and thus also with respect to hydrophobic proteins they may assist in partitioning to the polymer phase.

Partitioning (Step c)).

The main rule is that essentially all hydrophobic proteins, such as integral membrane proteins, that have an affinity structures corresponding to an affinity ligand present in a conjugate incorporated in the phase system will be partitioned to the micelle phase. Hydrophobic proteins not having such affinity structures and hydrophilic proteins will partition to the polymer phase.

The binding between the affinity ligand and the affinity structure may depend on variables such as pH, temperature, presence of agents stabilising a binding conformation, salt concentration etc. Typically these variables are selected for an optimal binding during step (c). In the case binding between the affinity ligand and the affinity structure requires protonation and/or that a pH-dependent conformation of the protein is required for binding, then there is also an optimal pH-range for the partition.

The partition pattern of a hydrophobic protein (for instance a membrane protein) will depend on the partition coefficient of the conjugate. The partitioning coefficient for a conjugate may, for instance, be unfavourable for the desired partition of the desired hydrophobic protein. In these cases one often can follow general rules well known in the field for guiding a compound into a desired phase of a system containing two or more distinct aqueous phases. This is typically accomplished by properly incorporating partition-directing agents such as selected salts, buffers, detergents etc. See for instance the experimental part in which this has been illustrated. This also means that the partition of a conjugate and thus also of a particular protein will depend on a combination of factors such as polymer in the polymer phase, micelle-forming agent, buffer, added salts, presence or absence of positively or negatively charged detergents etc. Both kinds and concentrations of these agents may influence the partition coefficient for a particular conjugate.

In case two or more conjugates that differ with respect to kind of affinity ligand are present in the phase system, two or more different hydrophobic proteins, for instance different membrane proteins, may simultaneously be partitioned to the polymer phase in step (c).

Optional Steps Subsequent to the Partitioning Step.

The hydrophobic protein(s) that is/are partitioned to the polymer phase collected in this step may be further purified, for instance by one or more extra separations in phase systems comprising two or more aqueous phases, electrophoresis, liquid chromatography (ion exchange, bio-affinity etc chromatography), precipitation, density gradient centrifugation, extraction, etc. Before additional purification steps it is often appropriate to release the protein complexed with the conjugate. This may be done in several ways:

(1) the pH may be changed to a pH at which affinity binding is low if the binding is pH-dependent;

(2) the phase may be treated with a structural analogue to the ligand;

(3) the temperature may be changed if the binding is temperature-dependent,

(4) the affinity structure may be cleaved off, for instance enzymatically, if the hydrophobic protein has been so designed,

(5) change in ionic strength (salt concentration) if the binding depends on ionic strength,

(6) addition of structure breaking agents, for instance hydrogen-bond breaking agents if the binding involves hydrogen bonds or the protein conformation needed for binding depends on hydrogen bonds, etc.

Release of the hydrophobic protein from the complex with the conjugate may be combined with mixing the collected polymer-enriched phase with a micelle-forming agent as defined above, thereby forming a phase system comprising two aqueous phase. If needed additional polymer may be added. By doing this the released hydrophobic protein(s), for instance one or more membrane proteins such as one or more integral membrane proteins is(are) partitioned to the newly formed micelle phase. The remaining polymer phase including the conjugate between the affinity ligand and the polymer may be recirculated into process step (b). Before this kind of recirculation, it may be advantageous to remove hydrophilic proteins retained in the phase in a previous cycle.

As an alternative the hydrophobic protein(s) upon release may be transferred to liposomes.

In the case two or more conjugates that differ with respect to kind of affinity ligand are present in the phase system, the corresponding proteins may be released separately and partitioned to a respective polymer phase. An alternative way is to use the conjugates one by one. This means that step c (c1) is carried out with a conjugate having one particular affinity ligand. Hydrophobic proteins not binding to the affinity ligand will remain in the micelle phase. Subsequently step c is then repeated (c2) by forming a new phase system comprising the micelle phase from step c1 plus a new fresh polymer phase plus a conjugate having an affinity ligand that differ from the affinity ligand used in step c1. Further repetitions may be carried out (steps c3, c4 etc) The polymer phase from each of the steps c1, c2, c3, c4 etc may then be treated as described above for step.

The invention will now be illustrated by the experimental part in which the best mode contemplated at the priority date is given. The invention is further defined in the attached patent claims.

### Experimental Part

Materials and Methods

Chemicals: Dextran T500 was purchased from Amersham Pharmacia Biotech (Uppsala, Sweden). Poly(ethylene glycol) (PEG) 40 000 was purchased from Serva (Heidelberg, Germany). C₁₂EO₅ (penta ethylene glycol mono-n-dodecyl ether) was obtained from Nikko Chemicals Co., (Tokyo, Japan). Triton X-100 (octylphenolpoly(ethyleneglycol ether)_{9,6}), was purchased from Sigma Chemicals Co. (St. Louis, Mo., USA). SDS (sodium dodecyl sulphate) was purchased from Merck (Darmstadt, Germany). DTAB (dodecyltrimethylammonium bromide) was obtained from Tokyo Kasei (Tokyo, Japan). The detergents were used without further purification. All water used was of Millipore quality. All other chemicals were of analytical grade.

Synthesis of metal chelating polymers: The chelating dextran was a kind gift from Amersham Pharmacia Biotech (Uppsala, Sweden) and had been manufactured with allyldextran (Mw 150,000) as a precursor. PEG 40 000-IDA was prepared by a modified procedure described by Chung et al. (1994) (32). 50 g of PEG 40 000 was freeze dried to remove excess amount water. 100 ml thionyl chloride was purified by distillation and the fraction at 69° C. was collected. The dried PEG and the distilled thionyl chloride was mixed and refluxed for 5 hours. Excess amount of thionyl chloride was removed by distillation. 20 g PEG 40 000 chloride, 5 g iminodiacetic acid (IDA) and 0.5 g NaOH was dissolved in 100 ml methanol and was refluxed for 24 hours. Methanol was removed by distillation and the mixture was resolved in 500 ml water. Impurities were removed by ultrafiltration in a Minisette Membrane Cassettes system (cut-off 10 KDa) from Filtron Technology Corporation (Northborough, Mass., USA). The mixture was lyophilized to give the product PEG 40 000-IDA. The degree of substitution (DS) was determined by titration of the chelating polymer with CuSO₄ and recording the shift in absorbance at 600 nm. The chelating polymer was loaded with Cu2+ by addition of excess amount of CuSO₄. Excess amount of the metal ion was removed by ultrafiltration in a Minisette Membrane Cassettes system (cut-off 10 KDa) from Filtron Technology Corporation (Northborough, Mass., USA) to yield the final product of PEG 40 000-IDACu(II). The degree of substitution was 0.22 mole IDA per mole PEG for PEG 40 000-IDACu(II)) and 0.14 mole IDA per mole monomer sugar for allyl dextran T150-IDACu(II). In this study we used Cu²⁺, since the general binding strength of chelated metal ions towards histidines decreases in the order: Cu²⁺>Ni²⁺>Zn²⁺>Co²⁺ (28).

Proteins: Bacterial growth conditions and the preparation of membranes from E. coli cells were as described previously (33). The used bacterial strain was GO105 containing the plasmid PJRHISA, which encodes for a genetically modified cytochrome bo3 oxidase with a carboxyl-terminus histidine tag (affinity structure) on subunit II (CytBO3). The tag contained 6 histidines. The purified membranes (10 g) was solubilized for 20 minutes in 100 ml of a solution containing 1% dodecylmaltoside, 20 mM Tris/HCl, pH 7.5, 300 mM NaCl and 5 mM imidazole and was centrifuged at 20 000 G for 20 minutes.

The CytBO3 was purified from the solubilized membranes by an one step affinity chromatography system using Ni²⁺-NTA as a column medium from Qiagen (Chatsworth, Calif., USA). The solubilized membrane fraction was applied to a 65 ml bed volume that was equilibrated with 20 mM Tris/HCl buffer, pH 7.5 with 300 mM NaCl, 5 mM imidazole and 0.03% dodecyl maltoside. The column was washed with 3 bed volumes of the equilibration buffer to remove any non-specific binding enzyme. The sample was eluted with a linear imidazole gradient from 5-150 mM. The resulting chromatogram showed two clear populations of CytBO3 that were termed “low” and “high” imidazole based on the elution concentration, where the “low” imidazole peak is lacking the histidine tag.

The pure CytBO3 from the “high” imidazole fraction was used in the partitioning experiments after the detergent and buffer was exchanged as appropriate for each experiment. The detergent and buffer was exchanged by binding the protein to a metal chelating chromatography column, HiTrap™ chelating from Amersham Pharmacia Biotech AB (Uppsala, Sweden), loaded with Ni²⁺. The bound protein was washed with four column volumes of the new buffer containing appropriate detergent and was eluted from the matrix with 100 mM imidazole solution containing the new buffer and the appropriate detergent. Imidazole was removed by gel filtration on PD-10 column from Amersham Pharmacia Biotech AB (Uppsala, Sweden).

Phase systems for the partitioning experiments: Aqueous two-phase systems, with total weight of 0.5 g, were prepared by mixing protein solution containing the detergent with a premixed stock phase system containing appropriate concentration of appropriate micelle-forming agent, polymer, buffer and additives. Thin glass test tubes (diameter 6 mm, length 50 mm) were used. All concentrations used were calculated as weight percentages. The systems were incubated at 4° C. for at least 15 minutes and were carefully mixed again. Phase separation was speeded up by centrifugation at 1800 g in 3-4 min in a table top centrifuge at 4° C. The polymer and micelle phases were isolated using a syringe and diluted as appropriate for assay.

Protein partition coefficient, yield and protein determination: The phases were analyzed for their CytBO3 content by measuring the absorbance at 406 nm against a reagent blank which contained a sample from an appropriately diluted phase from a system without protein.

The partitioning of a substance in an aqueous two-phase system is described by the partition coefficient K, which is defined as K=CT/CB, where CT and CB are the equilibrium concentrations of the partitioned substance in the upper and lower phases, respectively. The total and phase volumes were determined by weighing in distilled water to the same height in the test tube after cleaning and drying the tube. The protein recovery (mass balances) was determined by calculating the total protein amount added to the system and the amount of protein found in the different phases. A recovery between 90-110% was accepted as satisfactory. All results are average values after partitioning of the protein in at least two equal systems.

### FIGURE LEGENDS

FIG. 1 describes the concept of affinity partitioning hydrophobic integral membrane proteins in an extraction system in which there are a micelle phase and a polymer phase. Mixtures of a micelle-forming agent, for instance a non-ionic detergent, and a non-compatible hydrophilic polymer separates into a micelle phase in equilibrium with a polymer phase. (a) Hydrophobic proteins, such as integral membrane proteins, can be solubilized and partially purified in the micelle phase. (b) Each of the hydrophobic proteins can be individually affinity partitioned to the polymer phase by including an affinity ligand coupled to the polymer partitioning to the polymer phase.

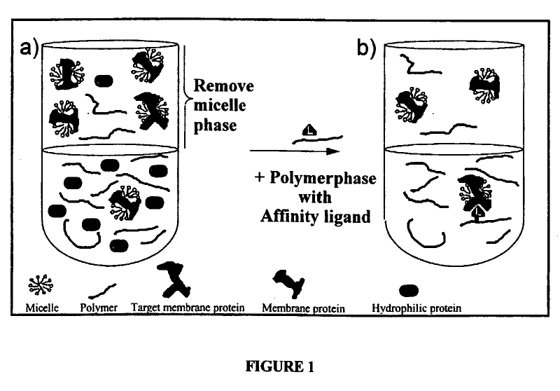

### EXAMPLE 1

### Effects of Conjugates Between IDA Chelated Copper and a Polymer (Dextran and PEG, Respectively) on the Partitioning of CytBO3

System Composition:

A. 6.5% (w/w) total PEG (polymer) concentration (i.e. PEG 40000 (100-X%)+PEG 40000-IDACu(II) (X=1, 5, 10, 20 and 50%) with DS of 0.22 mole IDA per mole PEG), 13.0% (w/w) Triton X-100 (micelle-forming agent), 10 mmol*kg−1 phosphate-borate buffer, pH 9.0, ca. 0.02% (w/w) CytBO3, temperature 3-4° C. (phase volume ratio 1.14). K>1 is equivalent to a preferred protein partitioning (the PEG phase is the upper phase).

B. 4.9% (w/w) C₁₂EO₅ (micelle-forming agent), 3.8% (w/w) total dextran (polymer) concentration (i.e. dextran T500+dextran T150-IDACu(II) with DS of 115 mole IDA per mole dextran), 10 mmol*kg−1 phosphate-borate buffer, pH 9.0, ca. 0.02% (w/w) CytBO3, temperature 3-4° C. (phase volume ratio 0.48). The concentration of Cu²⁺ varied from 0 to 2.9 mmol*kg⁻¹. K<1 is equivalent to a preferred protein partitioning (the dextran is the lower phase).

C. Similar systems was also set up for PEG/octylglucoside and PEG/dodecyl maltoside with PEG-IDA-Cu(II) as conjugate.

Results and discussion: Pure poly(histidine) tagged CytBO3 was increasingly partitioned into the polymer phase (upper phase) in a PEG/Triton X-100 system with increasing amount of PEG-IDA-Cu(II). The partitioning was thereby shifted from a partitioning in the micelle phase (K value of 0.015) to a partitioning into the polymer phase (K-value of 4.8). Thus, the chelating polymer shifted the K-value of CytBO3 300 times. Qualitatively similar results were obtained for systems C. An efficient affinity partitioning seems to require an excess amount of chelated copper ions, i.e. 50-100 times more mole metal ion per mole CytBO3 was needed.

The model protein CytBo3 partitioned strongly into the micelle phase when no affinity polymer was included in the system.

### EXAMPLE 2

### Effect of pH on the Partitioning of CytBO3

System composition: 0.071 mmole*kg⁻¹ PEG-IDA chelated copper ions, 6.5% (w/w) total PEG concentration (i.e. 5.2% (w/w) PEG 40000+1.3% (w/w) PEG 40000-IDACu(II) with DS of 0.22 mole IDA per mole PEG), 13.0% (w/w) Triton X-100, 10 mmol* kg⁻¹ phosphate-borate buffer, pH 5.5, 6.0, 6.5, 7.0, 7.5, 8.0, 8.5, 9.0, ca. 0.02% (w/w) CytBO3, temperature 3-4° C. (phase volume ratio 1.14). K>1 is equivalent to a preferred protein partitioning into the polymer phase (the PEG phase is the upper phase).

Results and discussion: The affinity partitioning of CytBO3 to the polymer phase increased with pH. At low pH the membrane protein partitioned strongly into the micelle phase, while the partitioning towards the polymer phase was strongly increased at increasing pH and leveled off around pH 7.5. The pH dependence can be explained by the deprotonation of the imidazole group of the histidine, which is completed at pH 7.5 (35,36). The effect of pH was independent of buffer used. The same effects were obtained in Tris/HCl buffer as in phosphate, phosphate-borate and sodium carbonate buffers for systems containing PEG-IDA-Cu(II). However, the partitioning of proteins in aqueous two-phase systems will be generally affected by addition of different salts and buffer ions (6,7). This pH dependence of metal affinity partitioning is convenient since the target protein can be back-extracted from the polymer phase into a micelle phase by decreasing the pH, and this can be done in suitable pH range for proteins. It also allows the possibility to remove and recycling the affinity polymer.

### EXAMPLE 3

### Effect of NaClO₄ Concentration on the Partitioning of CytBO3

System composition: 4.9% (w/w) C₁₂EO₅, 3.8% (w/w) total dextran concentration (i.e. dextran T500 (3.8 or 3.04%(w/w))+dextran T150-IDACu(II) with DS of 115 mole IDA per mole dextran (0 or 0.76% (w/w), respectively), 10 mmol*kg⁻¹ phosphate-borate buffer, pH 9.0, ca. 0.02% (w/w) CytBO3, temperature 3-4° C. (phase volume ratio 0.48). The concentration of NaClO₄ was varied between o to 100 mmole*kg⁻¹. K<1 is equivalent to a preferred protein partitioning (dextran phase is the lower phase).

Results and discussion: The effect on the partitioning of CytBO3 by dextran-IDA-Cu(II) was very low in systems with no addition of salt, K-values only shifted from 10 to 4 in a C₁₂EO₅/dextran system. Thus, the dextran could not pull the membrane protein sufficiently well into the polymer phase. But we also noted that the dextran-IDA-Cu(II) seemed to partition almost evenly between the phases (observed visually by the blue colour of the copper ions). Thus, the chelating polymer metal-ion complex preferred the micelle phase rather than to partition to the dextran enriched phase. This could be due to a negative net charge on dextran-IDA-Cu(II). The original buffer used was a sodium phosphate borate solution (pH 9.0). Both phosphate (HPO₄²⁻/H₂PO₄⁻) and borate ion are relatively hydrophilic in the Hofmeister series and have been found to partition into the dextran phase in a PEG/dextran system (6). The same behaviour can be expected in C₁₂EO₅/dextran system, due to a chemical resemblance between PEG/dextran and C₁₂EO₅/dextran systems. Thus, this uneven salt distribution will lead to the formation of an electrostatic potential difference between the two phases, which will influence the partitioning of molecules (37,38). In C₁₂EO₅/dextran system containing a phosphate/borate buffer a negatively charged molecule will prefer the micelle phase. In order to prove this an excess amount of sodium perchlorate (10 times the phosphate/borate amount) was added to the system. The blue color of dextran-IDA-Cu(II) was now strongly partitioned into the lower polymer phase (observed visually). Perchlorate ion (ClO₄⁻) is chaotropic and relatively hydrophobic in the Hofmeister series and will therefore have the opposite effect on charged molecules than phosphate/borate ions (37). Addition of sodium perchlorate also had a large impact on the affinity partitioning of CytBO3. Small amounts of added salt (10 mmole*kg⁻¹) could direct CytBO3 to the polymer phase (the K-value shifted with a factor of around 20). This effect is due to the shift in chelating polymer partitioning and not to the salt effect on the protein partitioning. In order to increase the partitioning of the recombinant poly(histidine) tagged integral membrane proteins into the polymer phase some different salts were tested (Table 1). The largest effect, i.e. lowest K-value, was obtained with sodium perchlorate (K=0.18) which was slightly better than sodium chloride (K=0.32).

### EXAMPLE 4

### Effect of an Ionic Detergent (SDS) on the Affinity Partitioning of CytBO3

System composition: 5.2% (w/w) C₁₂EO₅, 6.6% (w/w) total dextran concentration (i.e. dextran T500 (3.8 or 3.04%(w/w))+dextran T150-IDACu(II) with DS of 115 mole IDA per mole dextran (0 or 0.76% (w/w), respectively), 10 mmol*kg⁻¹ phosphate-borate buffer, pH 9.0, ca. 0.02% (w/w) CytBO3, temperature 3-4° C. (phase volume ratio 0.27). The SDS concentration varied from 0 to 0.25% (w/w). K<1 is equivalent to a preferred protein partitioning (the dextran phase is the lower phase).

Results and discussion: Protein partitioning can be shifted by addition of charged phase components to the system. For example, addition of ionic surfactants to the system, such as SDS or DTAB, creates a weakly charged mixed micelle that attracts oppositely charged proteins and repel similar charged proteins to the opposite phase (26), the effect is larger for hydrophilic proteins than for membrane proteins (15). Ionic detergents can also be used to direct the chelating dextran copper complex into the polymer phase and thereby the target protein. Small amounts of added anionic detergent SDS gave qualitatively similar results as addition of NaCl and NaClO₄. However, a relatively strong effect on the partitioning of CytBO3 was obtained by the addition SDS in systems without chelating polymer, which could be partitioned into the polymer phase (K-value of 0.8). Most proteins are net negatively charged around the optimal pH range (7.5-8.0) for metal affinity partitioning. They will therefore tend to partition more into the polymer phase with addition both of NaClO₄/NaCl and SDS, respectively. However, since the effect on membrane protein partitioning is larger for addition of SDS than salt it is advantageous to use salts to direct the partitioning of the metal chelating polymer into the polymer phase. Both the salt and ionic detergent experiments clearly indicate that the dextran-IDACu(II) is net negatively charged.

### EXAMPLE 5

### Effect of Imidazole Concentration on Partitioning of CytBO3

System composition: 0.071 mmole*kg⁻¹ PEG-IDA chelated copper ions, 6.5% (w/w) total PEG concentration (i.e. 5.2% (w/w) PEG 40000+1.3% (w/w) PEG 40000-IDACu(II) with DS of 0.22 mole IDA per mole PEG), 13.0% (w/w) Triton X-100, 10 mmol*kg⁻¹ phosphate-borate buffer, pH 9.0, ca. 0.02% (w/w) CytBO3, temperature 3-4° C. (phase volume ratio 1.14). The imidazole concentration varied from 0 to 50 mmole*kg⁻¹. K>1 is equivalent to a preferred protein partitioning (the PEG phase is the upper phase).

Results and discussion: Imidazole strongly competes with the binding between the protein and the metal chelate and high concentrations of imidazole are commonly used to elute the bound proteins in IMAC. This can also be used in metal chelating detergent/polymer system to back-extract the protein from the polymer phase. Imidazole at 1 mM was enough to release the bound protein in detergent/polymer aqueous two-phase systems. With 10 mM imidazole the protein was completely partitioned into the lower micelle phase with K=0.01, i.e. with 7000 times excess amount of imidazole (mole) compared to protein. Thus, addition of imidazole can be used in detergent/polymer systems for back-extraction of CytBO3 as an alternative to the use of low pH. Addition of low amount imidazole might also be used for reducing non-specific partitioning into the polymer phase in similar fashion as in IMAC.

### EXAMPLE 6

### Additions of Various Salts and Buffers on the Partitioning of CytBO3

The extra addition of salt to the system may be avoided by use of buffers that can direct the chelating dextran towards the polymer phase. Therefore, some different buffers with buffer capacity in the appropriate pH range were screened (Table 2). Many buffers can direct the dextran-IDACu(II) and the target protein sufficiently well into the polymer phase with K-values around 0.5, such as TES, Tris, MOPS, HEPES, and piperazine. No significant difference between the tested buffers was found with one exception, Tricine which just as phosphate/borate buffers was not able to partition CytBO3 into the metal chelating polymer phase. Thus, the tested buffers, which often are referred to as “good” buffers are also good buffers for metal affinity partitioning in dextran containing detergent/polymer aqueous two-phase systems. However, the partitioning of CytBO3 to the polymer phase was enhanced by addition of sodium perchlorate even in the case of MOPS and TES containing affinity systems (Table 2).

TABLE 1
Effect of different salts on the affinity partitioning
of CytBO3.
System composition: 1.90 mmole*kg⁻¹ allyldextran T150-IDA
chelated copper ions (0.76% (w/w) of the affinity polymer), 4.9%
(w/w) C₁₂EO₅, 3.8% (w/w) total dextran concentration and 10
mmole*kg⁻¹ phosphate-borate buffer pH 9.0, ca. 0.02% (w/w)
CytBO3, temperature 3-4° C. (phase volume ratio 0.48). Addition of
either 100 mmole*kg⁻¹ salt or 0.074% (w/w) ionic detergent. K <
1 is equivalent to a partitioning into the polymer phase.

| Salt | K_{additive} |
|---|---|
| NaClO₄ | 0.12 |
| NaCl | 0.32 |
| NaBr | 0.31 |
| NaSCN | 0.37 |
| Na(H₂PO₄²⁻/HPO₄²⁻), pH 9.0 | 0.70 |
| N⁺ (C₄H₉)₄(H₂PO₄⁻/HPO₄²⁻), pH 9.0 | 3.3 |
| SDS | 0.25 |
| DTAB | 60 |

TABLE 2
Effect of different buffers on the affinity partitioning
of CytBO3 and addition of NaClO₄.
System composition: 4.9% (w/w) C₁₂EO₅, 3.8% (w/w) total dextran
concentration and 20 mmole*kg⁻¹ buffer pH 7.5, ca. 0.02% (w/w)
CytBO3, temperature 3-4° C. (phase volume ratio 0.48). System with
affinity ligand contained 0.48 mmole*kg⁻¹ allyldextran T150-IDA
chelated copper ions (0.19% (w/w) of the affinity polymer).
Affinity system with salt addition contained 100 mmole*kg⁻¹ NaClO₄
(Kaff + NaClO₄). K < 1 is equivalent to a partitioning into the
polymer phase.

| | Partition coefficient | | |
|---|---|---|---|
| Buffer | Kₙₒ ligand | K_{aff} | K_{aff} + NaClO₄ |
| Sodium phosphate | 10.0 | 1.22 | — |
| Sodium phosphate- | 7.9 | 1.49 | — |
| borate | | | |
| Tricine | — | 3.9 | — |
| MOPS | — | 0.63 | 0.19 |
| HEPES | — | 0.60 | — |
| TES | — | 0.58 | 0.12 |
| TRIS | — | 0.46 | — |
| Piperazine | — | 0.39 | — |

### EXAMPLE 7

### Affinity Purification of an Integral Membrane Protein (His)6 Cytochrome bo3 Ubiquinol Oxidase (CytBO3) from E. Coli Membranes by Metal Affinity Partitioning in Micellar Extraction Systems

Materials and Methods: Membranes: Purified E. coli membranes. Target Protein: (His)6cytochrome bo3 ubiquinol oxidase (CytBO3). Chemicals: See previous examples for explanation and origin. Assay: Absorbance measured at 406 nm (CytBO3), BCA method (Total protein)

Experimental Set Up:

Solubilization step. E. coli membranes (20% wt.) was solubilized in a C₁₂EO₅/dextran T500 two phase system for 15 minutes at 4° C. by mixing on a wipeboard. Total system concentration was 7.9% C₁₂EO₅ and 6.0% dextran T500, 50 mM HEPES buffer pH 8.0. The solubilized proteins was partially purified by formation of a two-phase system which was enhanced at 40 000 G for 15 minutes at 4° C. The red top phase contained the main part of the CytBO3.

Washing steps: The membrane protein phase was removed by a syringe and transferred to a pure polymer phase (9.36% dextran T500, 0.22% C₁₂EO₅, 50 mM HEPES pH 8.0) of approximately the same size as the polymer phase in the first step. The system was mixed carefully and then separated into two phases in a centrifuge at 1800 G for 3-4 minutes. The phases were separated with a syringe and the procedure was repeated 2 times. CytBO3 remained in the top micelle phase while contaminating protein was washed away in the lower polymer phase.

Affinity step: The micelle phase containing the CytBO3 was removed by a syringe and was transferred to a new test-tube containing an affinity polymer phase. The affinity polymer phase contained 0.22% C₁₂EO₅, 50 mM HEPES buffer, pH 8.0, 200 mM NaClO4 and different amount of metal chelating allyldextran T150-IDA-Cu(II) (degree of substitution 0.14) mixed with dextran T500 to a total polymer concentration of 9.36% wt. The system was mixed and separated into two phases in a centrifuge at 1800 G for 3-4 minutes at 4° C. CytBO3 is then obtained in the lower polymer-enriched phase. Between all centrifugation step the protein was kept on ice.

Results:

See table 3.

TABLE 3

| | | Purif. | |
|---|---|---|---|
| | K_{CytBO3} | Factor | Yield |
| Membranes | — | 1 | 100 |
| Sol. Step micelle phase | 8.7 | 1.9 | 74 |
| Wash step 2 micelle phase | 9.9 | 2.4 | 60 |
| Wash step 3 micelle phase | 22 | 2.7 | 54 |
| Wash step 4 micelle phase | 23 | 3.3 | 37 |
| Aff. Step 5 polymer phase | 0.1 | 4.1 | 27 |
| Metal chelating | | 5.7 | 21 |
| chromatograph | | | |

Membranes 8.0 g/ml CytBO3; 56 mg/ml protein

### REFERENCE LIST

[1] G. von Jagow, H. Schägger, A practical guide to membrane protein purification, Academic Press, New York, 1991.

[2] S. Hjertén, H. Pan, K. Yao, in H. Peeters (Editor), Protides of the biological fluids. 29th Colloquium 1981, Pergamon Press, New York, 1982, p.15.

[3] J. E. Walker, M. Saraste, Curr. Opin. Struct. Biol. 6 (1996) 457. [4] A. Helenius, K. Simons, Biochim. Biophys. Acta 415 (1975) 29.

[5] L. M. Hjelmeland, in M. D. Deutcher (Editor), Methods in Enzymology 182, Academic Press, New York, 1990, p. 253.

[6] P.-\dot{A}. Albertsson, Partition of Cell Particles and Macromolecules, Wiley, New York, 1986.

[7] H. Walter, G. Johansson (Editors), Methods in Enzymology 228, Academic Press, New York, 1994.

[8] P.-\dot{A}. Albertsson, B. Andersson, J. Chromatogr. 215 (1981) 131.

[9] P.-\dot{A}. Albertsson, Biochem 12 (1973) 2525.

[10] C. Bordier, J. Biol. Chem. 256 (1981) 1604.

[11] A. Sánchez-Ferrer, R. Bru, F. García-Carmona, Crit. Rev. Biochem. Molecular Biol. 29 (1994) 275.

[12] W. L. Hinze, E. Pramauro, Crtit. Rev. Anal. Chem. 24 (1993) 133.

[13] T. Saitoh, H. Tani, T. Kamidate, T. Kamataki, H. Watanabe, Anal. Sci. 10 (1994) 299.

[14] H. Tani, T. Saitoh, T. Kamidate, T. Kamataki, H. Watanabe, Biotech. Bioeng. 56 (1997) 311.

[15] U. Sivars, F. Tjerneld, Biochim. Biophys. Acta (1999) 1417 (2000) 133.

[16] M. Yamazaki, M. Ohshika, T. Ito, Biochim. Biophys. Acta 1063 (1991) 175.

[17] S. M. Clegg, P. A. Williams, P. Warren, I. D. Robb, Langmuir 10 (1994) 3390.

[18] L. Piculell, K. Bergfeldt, S. Gerdes, J. Phys. Chem. 100 (1996) 3675.

[19] K. Zhang, G. Karlström, B. Lindman, J. Phys. Chem. 98 (1994) 4411.

[20] N. K. Pandit, J. Kanjia, Int. J. Pharm. 141 (1996) 197.

[21] N. K. Pandit, J. Kanjia, K. Patel, D. K. Pontikes, Int. J. Pharm. 122 (1995) 27.

[22] C. Wormuth, Langmuir 7 (1991) 1622.

[23] I. D. Robb, P. A. Williams, P. Warren, R. Tanaka, J. Chem. Soc. Faraday Trans. 91 (1995) 3901.

[24] N. Garg, I. Yu. Galaev, B. Mattiasson, Biotechnol. Appl. Biochem. 20 (1994) 199.

[25] T. Saitoh, W. L. Hinze, Talanta 42 (1995) 119.

[26] U. Sivars, K. Bergfeldt, L. Piculell, F. Tjerneld, J. Chromatogr. B 680 (1996) 43.

[27] H. Tani, T. Ooura, T. Kamidate, T. Kamataki, H. Watanabe, J. Chromatogr. B 708 (1998) 294.

[28] J. Porath, Protein Expression Purif. 3 (1992) 263.

[29] A. Otto, G. Birkenmeir, J. Chromatagr. 644 (1993) 25.

[30] D. Schustolla, W.-D. Deckwer, K. Schügerl, H. Hustedt, Bioseparation 3 (1992) 167.

[31] S.-S. Suh, F. H. Arnold, Biotech. Bioeng. 35 (1990) 682.

[32] B. H. Chung, D. Baily, F. H. Arnold, in H. Walter, G. Johansson (Editors), Methods in Enzymology Vol 228, Academic Press, New York, 1994, p. 167.

[33] J. E. Morgan, M. I. Verkhovsky, A. Puustinen, M. Wikström, Biochemistry 34 (1995) 15633.

[34] P. A. Maher, S. J. Singer, Proc. Nat. Acad. Sci. USA 82 (1985) 958.

[35] R. D. Johnson, R. J. Todd, F. H. Arnold, J. Chromatogr. A 725 (1996) 225.

[36] J. C. Cameselle, J. M. Ribeiro, A. Sillero, Biochem. Educ. 14 (1996) 131.

[37] G. Johansson, Acta Chem. Scand. B 28 (1974) 873.

[38] A. Pfennig, A. Schwerin, Ind. Eng. Chem. Res. 37 (1998) 3180.

[39] F. Tjerneld, J. Persson, and H. O. Johansson, WO 0040598.

[40] H. Ageland, L. Nyström and F. Tjerneld; International patent application WO 9811127.

[41] F. Tjerneld and G. Johansson, U.S. Pat. No. 4,7409,304.

[42] F Guinn, U.S. Pat. No. 5,907,035.

1. A method for separating one or more hydrophobic proteins, for instance membrane proteins such as integral membrane proteins, from a mixture of proteins, characterized in that said mixture is partitioned in a phase system comprising a micelle-enriched aqueous phase (micelle phase) and a polymer-enriched aqueous phase (polymer phase), at least part of a polymer of the polymer phase carrying an affinity ligand (polymer-affinity ligand conjugate) that is capable of binding to an affinity structure on at least one of said one or more hydrophobic proteins.

2. The method of claim 1, characterized in that the affinity structure is native for said at least one of said one or more hydrophobic proteins.

3. The method of anyone of claims 1-2, characterized in that the affinity ligand is a metal chelate.

4. The method of anyone of claims 1-3, characterized in that the affinity structure is not native to said at least one of said one or more hydrophobic proteins.

5. The method of anyone of claims 1-4, characterized in that at least one of a buffer, a salt or an ionic detergent supporting depletion of the conjugate from the micelle phase has been incorporated into the phase system and thereby also depletion of said at least one of said one or more hydrophobic proteins from the micelle phase.

6. The method of anyone of claims 1-5, characterized in that
a) the polymer phase is collected after partitioning and allowed to form a second phase system comprising a second micelle phase and a second polymer phase, and
b) the conditions are changed such that the binding between the affinity ligand and the affinity structure is inhibited thereby promoting partitioning of said at least one of said one or more hydrophobic proteins to said second micelle phase which is subsequently collected whereupon said one or more hydrophobic proteins if necessary may be further purified therefrom.

7. The method of anyone of claims 1-5, characterized in that the polymer phase is collected after partitioning, that the conditions are changed such that the binding between the affinity ligand and the affinity structure is inhibited, and that said at least one of said one or more hydrophobioc proteins thereupon is incorporated into liposomes.

8. The method of anyone of claims 6-7, characterized in that the binding has been inhibited by at least one of:
(a) changing pH;
(b) treating with a structural analogue to the ligand;
(c) changing temperature;
(d) cleaving off the affinity structure from said one or more hydrophobic proteins;
(e) changing the ionic strength (salt concentration),
(f) adding a structure breaking agent.
